(19) United States
(12) Patent Application Publication
(10) Pub. No.: US 2023/0132556 A1
(43) Pub. Date: May 4, 2023

(54) MICROFLUIDIC CHIP CELL SORTING AND TRANSFECTION

(71) Applicant: HEWLETT-PACKARD DEVELOPMENT COMPANY, L.P., Spring, TX (US)

(72) Inventors: Viktor SHKOLNIKOV, Palo Alto, CA (US); Daixi XIN, Palo Alto, CA (US)

(73) Assignee: HEWLETT-PACKARD DEVELOPMENT COMPANY, L.P., Spring, TX (US)

(21) Appl. No.: 17/911,559

(22) PCT Filed: Apr. 7, 2020
(86) PCT No.: PCT/US2020/026989

§ 371 Date: Sep. 14, 2022

(51) Int. Cl. C12M 1/42; C12N 13/00

(52) U.S. Cl. CPC C12M 35/02; C12N 13/00

(57) ABSTRACT

A cell transfection apparatus may include a microfluidic chip. The microfluidic chip may include a fluid input port, a cell sorter to sort target cells from non-target cells in fluid received through the input port, a cell transfection region comprising a single cell electroporation region to receive the target cells sorted from the nontarget cells and a fluid ejector to dispense a transfected target cell received from the cell transfection region.

### BACKGROUND

Transfection is sometimes used to engineer or modify biological cells for immunotherapies such as CAR-T therapy. During transfection, nucleic acids and small proteins are introduced into eukaryotic cells. Electroporation is sometimes used to electrically open pores in the cell membrane for the introduction of the nucleic acids and small proteins.

### BRIEF DESCRIPTION OF THE DRAWINGS

FIG. 1 is a schematic diagram illustrating portions of an example cell transfection apparatus.

FIG. 2 is a flow diagram illustrating an example cell transfection method.

FIG. 3 is a schematic diagram illustrating portions of an example cell propagation system comprising the cell transfection apparatus of FIG. 1.

FIG. 4 is a schematic diagram illustrating portions of an example cell transfection apparatus.

FIG. 5 is a schematic diagram illustrating portions of an example cell transfection apparatus.

FIG. 6 is a schematic diagram illustrating portions of an example cell transfection apparatus.

FIG. 6 is a schematic diagram illustrating portions of an example cell transfection apparatus.

FIG. 7 is a schematic diagram illustrating portions of an example cell transfection apparatus.

FIG. 8 is a schematic diagram illustrating portions of an example cell transfection apparatus.

Throughout the drawings, identical reference numbers designate similar, but not necessarily identical, elements. The FIGS. are not necessarily to scale, and the size of some parts may be exaggerated to more clearly illustrate the example shown. Moreover, the drawings provide examples and/or implementations consistent with the description; however, the description is not limited to the examples and/or implementations provided in the drawings.

### DETAILED DESCRIPTION OF EXAMPLES

Disclosed are example cell transfection apparatus, cell transfection methods and cell propagation systems that provide for the enhanced generation of engineered biological cells. The example apparatus, methods and systems may provide for an automated integrated apparatus or system that reduces contamination, reduces operator error, and reduces labor cost. The example apparatus, method and systems may sort cells prior to transfection to improve homogeneity of the transfected cells which may result in improved testing and therapy outcomes.

The example cell transfection apparatus, example cell transfection methods and example cell propagation systems integrate a cell sorter, a cell transfection region and a fluid ejector on a single microfluidic chip. Such integration facilities a compact arrangement that may reduce cost and contamination issues. Such integration may facilitate a streamlined automated process for efficiently and economically generating engineered biological cells for therapeutic and academic purposes.

In some implementations, the sorted cells undergo electrotransfection, wherein electrodes in an electroporation region apply an electric field with an amplitude and voltage that induces momentary poration of the cell membrane to allow material to diffuse into the cell. Following the introduction of the material into the cell, the pores are allowed to close. In some implementations, the cells may be diluted and directed across a single cell electroporation region. Each of the cells sequentially passing through the electroporation region experiences same electric field are not perturbed by neighbor cell passing through electroporation region at the same time. This results in a more uniform and reliable transfection of cells.

In some implementations, cells are sequentially moved through the single cell electroporation region by pumping devices also integrated into the single microfluidic chip. For example, the pumping devices may comprise inertial pumps. In some implementations, the inertial pumps may comprise thermoresistive electrodes which vaporize liquid to create a bubble which displaces and pumps fluid.

In some implementations, cells are sequentially pulled or drawn through the single cell electroporation region by a fluid ejector integrated into the single microfluidic chip. Ejection of fluid by the fluid ejector creates a fluid pressure differential to draw cells, suspended in fluid, across the electroporation region. In some implementations, the fluid ejector may comprise a fluid actuator which displaces fluid through an ejection orifice to dispense the sorted and transfected cells.

As noted above, the integration of the cell sorting, cell transfection and fluid ejection into a single microfluidic chip provides a compact and cost-effective solution for generating engineered cells. The compact microfluidic chip may be well adapted for incorporation into a larger, but more compact and cost-effective, cell propagation system. The microfluidic chip may be incorporated into a cell propagation system that also includes a multi-well plate such that different samples of cells may be ejected or dispensed by the fluid ejector into different wells. Propagation of the different cells may be further enhanced through the addition of reagents and liftoff agents supplied by liquid handler. In some implementations, an imager or other sensor may also monitor the propagation of cells in the different wells. Due to compactness of the overall propagation system, it may be more readily housed within an incubator which controls environmental factors such as temperature, humidity and carbon dioxide levels for enhanced cell propagation.

The disclosed cell transfection apparatus, cell transfection methods and cell propagation systems may be well suited for use in chimeric antigen receptor (CAR) T cell therapy. In CAR T cell therapy, a sample of patient's T cells (part of the patient's immune system) are collected from blood and then modified or engineered to produce the special CAR constructs on their surface. Such T cells may be engineered to express disease-specific targets which then may be propagated to therapeutic levels before being infused back into the patient from which the cells were taken.

With the disclosed cell transfection apparatus, cell transfection methods and cell propagation systems, the T cells collected from the blood of the patient may first be sorted to separate naïve T cells from mature T cells. Through sorting, naïve T cells can be separated from mature T cells for transfection and propagation. The naïve T cells may have a higher therapeutic efficiency than the mature and activated T cells. As a result, transfected cells generated by the cell transfection apparatus, methods and systems may have a higher percentage of naïve T cells. The higher percentage of naïve T cells may lead to a higher drug persistence and enhanced therapeutic results.

Disclosed are example cell transfection apparatus that may include a microfluidic chip. The microfluidic chip may include a fluid input port, a cell sorter to sort target cells from non-target cells in fluid received through the input port, a cell transfection region comprising a single cell electroporation region to receive the target cells sorted from the nontarget cells and a fluid ejector to dispense a transfected target cell received from the cell transfection region.

Disclosed are example cell transfection methods may include depositing a solution into a port of a microfluidic chip, sorting a target cell from a non-target cell of the solution with a cell sorter on the microfluidic chip, moving the target cell through a single cell electroporation region of the chip, transfecting the electroporated target cell on the chip and dispensing the transfected cell from the chip.

Disclosed are example cell propagation system systems that may include a multi-well plate, a microfluidic chip, a liquid handler, a stage and a controller. The microfluidic chip may include a fluid input port to receive a fluid containing target cells, a cell transfection region comprising a single cell electroporation region and a fluid ejector to eject droplets of fluid to pull the solution containing the target cells through the single cell electroporation region. The liquid handler is to exchange media within wells of the well plate. The stages position the multi-well plate relative to the fluid ejector in the liquid handler. The controller may help control signals controlling stage, the fluid ejector in the liquid handler.

FIG. 1 is a block diagram schematically illustrating portions of an example cell transfection apparatus 20. Cell transfection apparatus 20 comprises a microfluidic chip 22 that integrates cell sorting, cell transfection and controlled cell ejection or dispensing into a compact arrangement that may reduce cost and contamination issues. Such integration may facilitate a streamlined automated process for efficiently and economically generating engineered biological cells for therapeutic and academic purposes.

Microfluidic chip 22 may comprise a platform or substrate formed by a single layer multiple layers that support or form microfluidic passages, chambers and volumes and that further support electronic elements in the form of transistors, resistors, fluid actuators and their associated electrical conductive wires or traces. The platform or substrate may comprise a silicon-based wafer or other such similar materials used for microfabricated devices (e.g., glass, gallium arsenide, quartz, sapphire, metal, plastics, etc.). In some implementations, microfluidic chip 22 may be formed from a glass reinforced epoxy laminate material such as a glass epoxy laminate such as FR4, wherein microfluidic channels may be formed in the laminate material or may be formed in other structures mounted to the laminate material.

As will be appreciated, portions of microfluidic chip 22 may be formed by performing various microfabrication and/or micromachining processes on a substrate to form and/or connect structures and/or components. Microfluidic channels and/or chambers may be formed by performing etching, microfabrication processes (e.g., photolithography), or micromachining processes in a substrate. Accordingly, microfluidic channels and/or chambers may be defined by surfaces fabricated in the substrate of a microfluidic device. In some implementations, microfluidic channels and/or chambers may be formed by an overall package, wherein multiple connected package components combine to form or define the microfluidic channel and/or chamber.

In some examples described herein, at least one dimension of a microfluidic channel and/or capillary chamber may be of sufficiently small size (e.g., of nanometer sized scale, micrometer sized scale, millimeter sized scale, etc.) to facilitate pumping of small volumes of fluid (e.g., picoliter scale, nanoliter scale, microliter scale, milliliter scale, etc.). For example, some microfluidic channels may facilitate capillary pumping due to capillary force. In addition, examples may couple at least two microfluidic channels to a microfluidic output channel via a fluid junction.

Microfluidic chip 22 comprises fluid input port 30, cell sorter 34, cell transfection region 38 and fluid ejector 42. Fluid input port 30 comprises an opening or passage through which a volume of fluid may be introduced into microfluidic chip 22. The volume of fluid may contain cells for transfection. The fluid may also contain additional particles and other non-target cells. In some implementations, fluid input port is sized for receiving a pipette or needle by which the fluid may be supplied to microfluidic chip 22. In some implementations, fluid input port 30 may comprise a fluid coupling for connection to a tube supplying the fluid or connection to an output port of a fluid source. In some implementations, microfluidic chip 22 may be insertable into the fluid source, wherein the fluid input port becomes aligned with the output port of the fluid source.

Cell sorter 34 comprises a portion of microfluidic chip 22 that receives the fluid received through fluid input port 30 and that sorts target cells from non-target cells or particles in the fluid. Cell sorter 34 may distinguish between target cells and non-target cells based upon various differences between the target cells and nontarget cells. For example, cell sorter 34 may distinguish between target cells and non-target cells based upon size or using techniques such as negative or positive enrichment, dielectrophoretics or acoustics. As shown by broken lines, target cell 35, sorted by cell sorter 34, is pumped or otherwise moved to cell transfection region 38.

Cell transfection region 38 comprises a region of microfluidic chip 22 that carries out transfection with respect to the sorted target cell 35. Cell transfection region 38 comprises that apply an electric field with an amplitude and voltage that induces momentary poration of the membrane of target cell 35 to allow material to diffuse into the cell 35. For example, to transfect naïve T cells, transfection region 38 may apply an electric field having 0.01 V/um to 1V/um. In other implementations, the field may have other values depending upon the solution containing the cells. Following the introduction of the material into the cell, the pores are allowed to close.

In some implementations, the fluid containing the cell 35 may be diluted and directed across a single cell electroporation region sized such that cells move through the region in single file order, such that a single individual cell 35 undergoes electroporation and electrotransfection at a time. The fluid passage through which cell 35 moves or is contained during electro transfection is sized such that additional neighboring cells are inhibited from entering the same fluid passage and extending alongside, in parallel to, target cell 35 during electroporation and transfection. There are no neighboring cells which may interfere with or perturb the electric field being applied to the target cell 35. Thus, each of the cells 35 sequentially passing through the cell transfection region 38 and its electroporation region experiences same electric field. This results in a more consistent and uniform transfection of the cells being generated by apparatus 20. Although such sizes may vary depending upon the cells being engineered, examples of a cross-sectional area of region 38 include, but are not limited to. 5×10 um, 10×10 um, 20×20 um, 20×10 um, 50×50 um, etc. In some implementations where larger cells are to be transfected, the cross-sectional dimensions may be greater.

In some implementations, cell 35 is sequentially moved through the cell transfection region 38 by pumping devices also integrated into the single microfluidic chip 22. For example, the pumping devices may comprise inertial pumps. In some implementations, the inertial pumps may comprise thermoresistive electrodes which vaporize liquid to create a bubble which displaces and pumps fluid.

Fluid ejector 42 comprises a fluid actuator that dispenses or ejects the transfected target cell 35 through an ejection orifice. In one implementation, fluid ejector 42 may comprise a thermal resistor which, upon receiving electrical current, heats to a temperature above the nucleation temperature of the fluid so as to vaporize a portion of the adjacent fluid to create a bubble which displaces the fluid through the associated ejection orifice. In other implementations, the fluid ejector 42 may comprise other forms of fluid actuators. In other implementations, the fluid ejector 42 may comprise a fluid actuator in the form of a piezo-membrane based actuator, an electrostatic membrane actuator, mechanical/impact driven membrane actuator, a magnetostrictive drive actuator, an electrochemical actuator, and external laser actuators (that form a bubble through boiling with a laser beam), other such microdevices, or any combination thereof.

In some implementations, fluid ejector 42 dispenses the transfected target cell 35 into a target location, such as into a well of a multi-well plate. In some implementations, fluid ejector 42 dispenses fluid to pull or draw cell through the single cell transfection region 38. Ejection of fluid by the fluid ejector 42 creates a fluid pressure differential to draw cells, suspended in fluid, across the transfection region 38. In some implementations, fluid ejector 42 may be used alone or in combination with other pumps (such as inertial pumps) supported by chip 22 to sequentially move cells sorted by cell sorter 34 through and across cell transfection region 38.

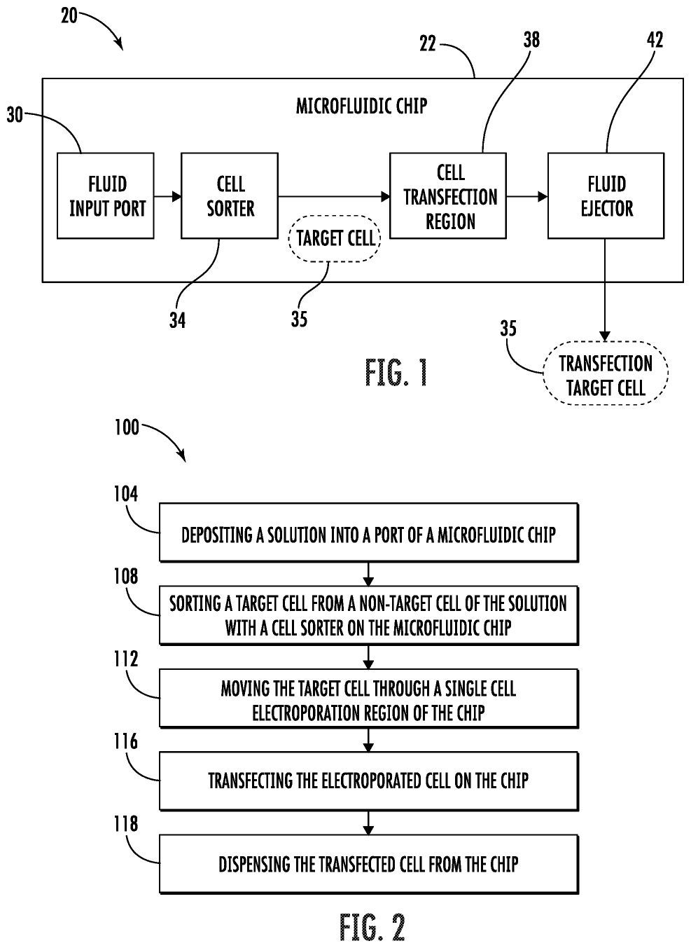

FIG. 2 is a flow diagram of an example cell transfection method 100. Method 100 facilitates a streamlined automated process for efficiently and economically generating engineered biological cells for therapeutic and academic purposes. Although method 100 is described in the context of being carried out by cell transfection apparatus 20, it should be appreciated that method 100 may likewise be carried out with any of the following described cell transfection apparatus and cell propagation systems or with similar cell transfection apparatus or cell propagation systems.

As indicated by block 104, a fluid solution is deposited into a port, such as fluid input port 30, of a microfluidic chip, such as microfluidic chip 22. The solution may contain target cells 35 which are to be transfected. In some implementations, the fluid solution may be deposited with a pipette or needle. In other implementations, the fluid solution may be deposited via a connection to the fluid input port.

As indicated by block 108, the target cell 35 is sorted from non-target cells of the fluid solution with a cell sorter, such as cell sorter 34, on the microfluidic chip 22. The sorting of the target cell or target cells from non-target cells in the fluid solution may be based upon size or using techniques such as negative or positive enrichment, dielectrophoretics or acoustics.

As indicated by block 112, those target cells which have been sorted or separated from other types of cells or other particles are moved through cell transfection region 38 of the chip 22. In some implementations, the target cells are pumped by pump located on the microfluidic chip 22. For example, inertial pumps may be formed on microfluidic chip 22 for moving the cells through the cell transfection region. In some implementations, fluid pressures created on the microfluidic chip through the ejection of fluid by fluid ejector may be used to pull or draw cells through and across the cell transfection region.

As indicated by block 116, while the cells are within the cell transfection region or are being moved through the cell transfection region, the cells undergo transfection. In some implementations, the cells undergo electro transfection or electroporation. During electrotransfection or electroporation, electrodes on the microfluidic chip 22 apply an electric field with an amplitude and voltage that induces momentary poration of the membrane of the individual target cell received within the cell transfection region to allow material to diffuse into the cell. Following the introduction of the material into the cell, the pores are allowed to close. As indicated by block 118, transfected cells are then dispensed from the microfluidic chip 22.

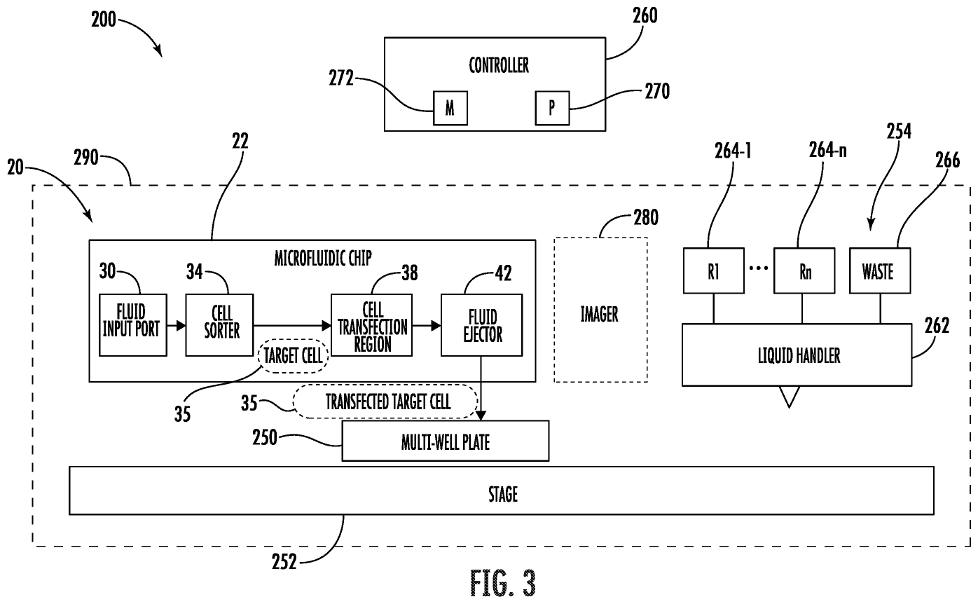

FIG. 3 is a diagram schematically illustrating portions of an example cell propagation system 200. Cell propagation system 200 facilitates the generation of engineered biological cells as well as the propagation of the engineered biological cells. In addition to microfluidic chip 22 (described above), cell propagation system 200 comprises multi-well plate 250, stage 252, media exchange system 254 and controller 260.

Multi-well plate 250 comprises an array of individual wells which are to receive the transfected cells dispensed by fluid ejector 42. Stage 252 comprises a motorized stage that controllably positions individual wells of multi-well plate 250 opposite to fluid ejector 42 for receiving a transfected cell or multiple transfected cells. Stage 252 may further position the individual wells of multi-well plate 250 opposite to media exchange system 254. In some implementations where microfluidic chip 22 and media exchange system 254 are themselves movable or positionable relative to multi-well plate 250, stage 252 may be omitted.

Media exchange system 254 exchanges fluid in each of the individual wells of multi-well plates 250 facilitate the propagation of the transfected cells, the growth in number or multiplication of transfected cells. Media exchange system 254 may further remove waste. Media exchange system 254 may comprise liquid handler 262, reagent sources 264-1-264-n (collectively referred to as reagent sources 264) and waste reservoir 266. Liquid handler 262 pumps or directs the flow of fluid to and from the individual wells of multi-well plate 250. Liquid handler 262 refreshes media for continuous cell growth. Liquid handler 262 supplies reagents from reagent sources 264 to the transfected cells contained within the wells of multi-well plate 250. Liquid handler 262 may further withdraw used or exhausted fluid from such wells. In some implementations, liquid handler 262 may comprise an automated pipetting system or peristaltic pump.

Controller 260 controls the operation of cell propagation system 200. Controller 260 comprises a processor 270 that follows instructions contained on a non-transitory computer-readable medium in the form of memory 272. Such instructions direct the processor 270 to output control signals controlling the pumping or movement of cells from cell sorter 34 through transfection region 38 and the ejection of fluid (with the suspended transfected cells) by fluid ejector 42. Such control signals may further control the operation of 252 to properly position individual wells of multi-well plate 250 opposite to fluid ejector 42 and to properly position the individual wells of multi-well plate 250 opposite to liquid handler 262. In some implementations, such control signals may further control the sorting of cells by cell sorter 34 and/or the pumping of fluid from input port 30 to cell sorter 34. Controller 260 may communicate with the other components of propagation system 200 in a wired or wireless manner.

As shown by broken lines, in some implementations, cell propagation system 200 may additionally comprise an imager 280. Imager 280 may monitor and inspect the cells within multi-well plate 250 during their propagation. Signals from imager 280 may be supplied to controller 260 which, pursuant to the instructions contained in memory 272, adjusts the exchange of media by media exchange system 254 and/or adjusts the sorting, transfection and dispensing operations carried out on microfluidic chip 22 based upon the signals from imager 280. Using the information acquired by imager 280, controller 260 may provide feedback control to enhance the transfection performance, cell viability and cell propagation. In some implementations, imager 280 may be omitted.

As further shown by broken lines, in some implementations, cell propagation system 200 may additionally comprise an incubator 290. Incubator 290 comprises an enclosure containing microfluidic chip 22, stage 252, multi-well plate 250 and media exchange system 254. In some implementations, incubator 290 may additionally contain imager 280, when provided. In other implementations, imager 280 may image the contents of multi-well plate 253 window or transparent portion of incubator 290.

Incubator 290 facilitates the control of the environment in which the cells are transfected and propagated. For example, incubator 290 may facilitate the control of temperature, humidity and carbon dioxide concentration levels to enhance the propagation of transfected cells. In some implementations, controller 260, following instructions contained in memory 272, may output control signals to heaters, humidifiers, vents, fans or carbon dioxide sources connected to the interior of incubator 290 to adjust the temperature, humidity and carbon dioxide levels within incubator 290. In some implementations, such adjustment may be based upon data obtained from imager 280. In some implementations, such adjustments may be based upon predetermined timing protocols or schedules. In some implementations, incubator 290 may be omitted.

In some implementations, cell propagation system 200 may be used for CAR T cell therapy. As part of the CAR T cell therapy, a sample of patient's T cells (part of the patient's immune system) are collected from blood and then modified or engineered to produce the special CARs on their surface. Such T cells may be engineered to express disease-specific targets which then may be propagated to therapeutic levels before being infused back into the patient from which the cells were taken.

With cell propagation system 200, the T cells collected from the blood of the patient are inserted to fluid input port 30 and then sorted by cell sorter 34 to separate naïve engineered T cells from active T cells. Through sorting, naïve T cells can be separated from active T cells. The fluid having the higher concentration of naïve T cells is then transmitted through cell transfection region 38 where the naïve T cells are transfected with CAR plasmids for disease specific targets. Once transfected, cells are dispensed by fluid ejector 42 into designated wells of multi-well plate 250. Thereafter, the wells containing the transfected cells are repositioned opposite to media exchange system 254 which exchanges cell growth media to enhance the growth or propagation of the transfected cells. The larger number of propagated transfected cells may then be reintroduced into the patient for therapeutic purposes.

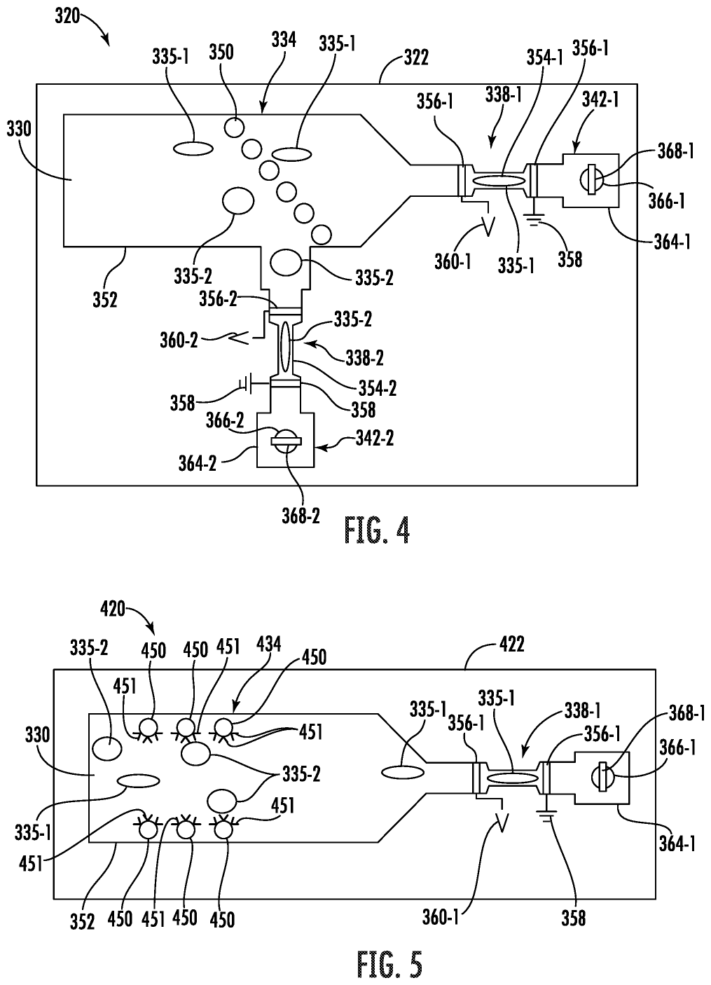

FIG. 4 schematically illustrates portions of an example cell transfection apparatus 320. Cell transfection apparatus 320 illustrates one example of how biological cells may be sorted by size for subsequent transfection and dispensing. Similar to cell transfection apparatus 20, cell transfection apparatus 320 is supported by or integrated into a single microfluidic chip 322. Microfluidic chip 322 is similar to microfluidic chip 22 except microfluidic chip 322 specifically comprises fluid input port 330, cell sorter 334, cell transfection regions 338-1, 338-2 (collectively referred to as transfection regions 338) and fluid ejector's 342-1, 342-2 (collectively referred to as fluid ejectors 342).

Fluid input port 330 is similar to fluid input port 30 described above. Fluid input port 330 comprises an opening or passage through which a volume of fluid may be introduced into microfluidic chip 322. The volume of fluid may contain cells for transfection. The fluid may also contain additional particles and other non-target cells. In some implementations, fluid input port 330 is sized for receiving a pipette or needle by which the fluid may be supplied to microfluidic chip 322. In some implementations, fluid input port 330 may comprise a fluid coupling for connection to a tube supplying the fluid or connection to an output port of a fluid source. In some implementations, microfluidic chip 322 may be insertable into the fluid source, wherein the fluid input port becomes aligned with the output port of the fluid source.

Cell sorter 334 sorts cells in the fluid received through input port 330 based upon the size-elasticity and/or shape changing characteristics of such cells. In the example illustrated, cell sorter 334 comprises an array of spaced pillars 350 extending from a floor to roof of fluid passage 352 and forming a filter, wherein pillars 350 are spaced apart from one another by a distance sufficiently large to allow first biological cells 335-1 to pass therebetween and sufficiently small to inhibit or block the passage of biological cells 335-2. The spacing between pillars 350 may be dependent upon the expected size of the different biological cells 335 as well as their potentially different elasticities or ability to compress or change shape. In some other implementations, the filter may be formed by horizontal bars extending side to side within passage 352. In other implementations, cell sorter 334 may comprise a filter or other arrangement of structures that permit the passage of some biological cells while blocking the passage of other biological cells based upon their different sizes.

Cell transfection regions 338 branch off of microfluidic passage 352 on opposite sides of the filter form by pillars 350. Cell transfection regions 338-1 and 338-2 comprise constricted fluid passages 354-1 and 354-2 (collectively referred to as passages 354), respectively. Passage 354-1 is sized to inhibit to biological cells 335-1 from moving through passage 354-1 in parallel. Rather, passage 354-1 is sized social force a sequential ordering of individual cells (single cells) through and across passage 354-1. Similarly, passage 354-2 is sized to inhibit to biological cells 335-2 from moving through passage 354-2 in parallel. Rather, passage 354-2 is sized so as to force a sequential ordering of individual cells (single cells) through and across passage 354-2.

In some implementations, passages 354-1 and 354-2 may have different cross-sectional dimensions and/or different cross-sectional shapes from one another based upon the particular cross-sectional dimensions, shape and/or shape changing characteristics of the respective cells 335-1 and 335-2 that are to be transfected in such regions. In some implementations where cell transfection apparatus 320 is used as part of CAR T therapy, the spacing between pillars 550 is sized so as to allow the passage of naïve T cells and block or inhibit the passage of active T cells.

Cell transfection region 338-1 further comprises a pair of electrodes 356-1 that are electrically connected to a ground 358 and a voltage source 360 such that electric field is created across passage 354-1 containing the single cell 335-1. The electric field thus formed has characteristics so as to electrically open pores in the membrane of the individual cell 335-1 contained within transfection region 338-1 for the introduction of the nucleic acids and small proteins.

Similar to cell transfection region 338-one, cell transfection region 338-2 further comprises a pair of electrodes 356-2 that are electrically connected to a ground 358 and a voltage source 360-2 such that electric field is created across passage 354-2 containing the single cell 335-2. The electric field thus formed has characteristics so as to electrically open pores in the membrane of the individual cell 335-2 contained within cell transfection region 338-2 for the introduction of the nucleic acids and small proteins. In some implementations, the electric fields created across transfection region 338-1 and 338-2 are different from one another due to the different characteristics of the cells being transfected within such different regions.

While within the cell transfection region 338, the cells 335 undergo electroporation, wherein pores in the membrane are opened. While within passages 354-1 and 354-2, cells 335-1 and 335-2 are transfected with foreign materials such as nucleic acids and small proteins. In implementations where apparatus 320 is used as part of CAR T therapy, the naïve T cells in cell transfection region 338-1 may be infused with CAR receptors.

Fluid ejectors 342-1 and 342-2 comprise ejection passages or chambers 364-1, 364-2 (collectively referred to as chambers 364), ejection orifices 366-1, 366-2 (collectively referred to as ejection orifices 366) and fluid actuator 368-1, 368-2 (collectively referred to as fluid actuator 368). Chambers 364-1, 364-2 of fluid ejectors 342 receive transfected cells from their respective transfection region 338-1, 338-2. Ejection office 366-1, 366-2 extend from their respective chambers 364-1, 364-2.

Fluid actuators 368-1, 368-2 comprise devices that displace fluid within their respective chambers 364-1, 364-2 through their respective ejection orifices 366-1, 366-2. Such fluid actuator 368 also dispense the transfected cells through the ejection orifices 366. In one implementation, each of fluid actuators 368 comprises a thermal resistor which, upon receiving electrical current, heats to a temperature above the nucleation temperature of the fluid so as to vaporize a portion of the adjacent fluid to create a bubble which displaces the fluid through the associated ejection orifice. In other implementations, each of fluid actuators 368 may comprise other forms of fluid actuators. In other implementations, the fluid ejector 42 may comprise a fluid actuator in the form of a piezo-membrane based actuator, an electrostatic membrane actuator, mechanical/impact driven membrane actuator, a magnetostrictive drive actuator, an electrochemical actuator, and external laser actuators (that form a bubble through boiling with a laser beam), other such microdevices, or any combination thereof.

In the example illustrated, upon receiving a transfected cell 335-1, fluid actuator 368-1 may be actuated to dispense eject fluid, containing the transfected cell 335-1, through ejection orifice 366-1, to an underlying target site, well, passage or other receptacle. In some implementations, the dispensing of fluid through ejection orifice 366-1 creates a fluid pressure differential which draws fluid and other suspended cells across pillars 350-1. The pressure differential may further draw an individual cell 335-1 into transfection region 338-1 for transfection.

In the example illustrated, upon receiving a transfected cell 335-2, fluid actuator 368-2 may be actuated to dispense or eject fluid, containing the transfected cell 335-2, through ejection orifice 366-2 to an underlying target site, well, passage or other receptacle. In some implementations, the dispensing of fluid through ejection orifice 366-2 creates a fluid pressure differential which draws fluid and other suspended cells across pillars 350-2. The pressure differential may further draw an individual cell 335-2 into transfection region 338-2 for transfection. In other implementations, additional inertial pumps may be provided in passage 352 or elsewhere for moving cells 335-1 through pillars 350 and across transfection region 338-1 or for moving cells 335-2 through transfection region 338-2.

In some implementations, transfection region 338-2 and fluid ejector 342-2 may be omitted. For example, in some implementations, fluid and cells not passing through the filter formed by pillars 350 may instead be directed to a waste receptacle or port. In some implementations, transfection region 338-2 may be omitted, wherein fluid and cells not passing through the filter formed by pillars 350 may be dispensed by fluid ejector 342-2 to a waste passage or reservoir. Although microfluidic chip 322 is illustrated as having a single filter, in other implementations, microfluidic chip 322 may comprise multiple different filters which are in series with one another, wherein three or more different biological cells may be separated or sorted from one another, wherein different cells that have been sorted from one another may undergo different transfection procedures or disposal.

FIG. 5 schematically illustrates portions of an example cell transfection apparatus 420. FIG. 5 illustrates an example of how a microfluidic chip may carry out the sorting of cells based upon affinity prior to transfection and dispensing by the same microfluidic chip. Cell transfection apparatus 420 is similar to cell transfection apparatus 320 described above except that cell transfection apparatus 420 comprises a microfluidic chip 422 comprising cell sorter 434 in place of cell sorter 334 and omitting transfection region 338-2 and fluid ejector 342-2. Those remaining components of cell transfection apparatus 420 which correspond to components of cell transfection apparatus 320 are numbered similarly.

Cell sorter 434 comprises pillars 450 or other structures which are enriched so as to attract non-target cells 335-2 while permitting target cells 335-1, which are less attracted to pillars 450, to pass by to transfection region 338-1. In some implementations, pillars 450 are enriched with anti-bodies 451 for attracting the nontarget cells 335-2 while allowing the target cells 335-1 to pass. In implementations where cell transfection apparatus 420 is used as part of a CAR T therapy, the antibodies are chosen so as to attract the active T cells, allowing naïve T cells to pass. Once sorted and separated from cells 335-2, the target cell 335-1 may be directed in a single file basis through and across cell transfection region 338-1 (described above). Once transfected, the cells 335-1 may be controllably and selectively dispensed through ejection orifice 366-1 by fluid actuator 368-1. In the example illustrated, the dispensing of fluid by fluid actuator 368-1 creates a negative pressure differential which draws or pulls cell 335-1 through and across transfection region 338-1 in a single file order. In other implementations, inertial pumps may be provided for assisting with the movement of cells 335-1 through transfection region 338-1 to ejector 342-1.

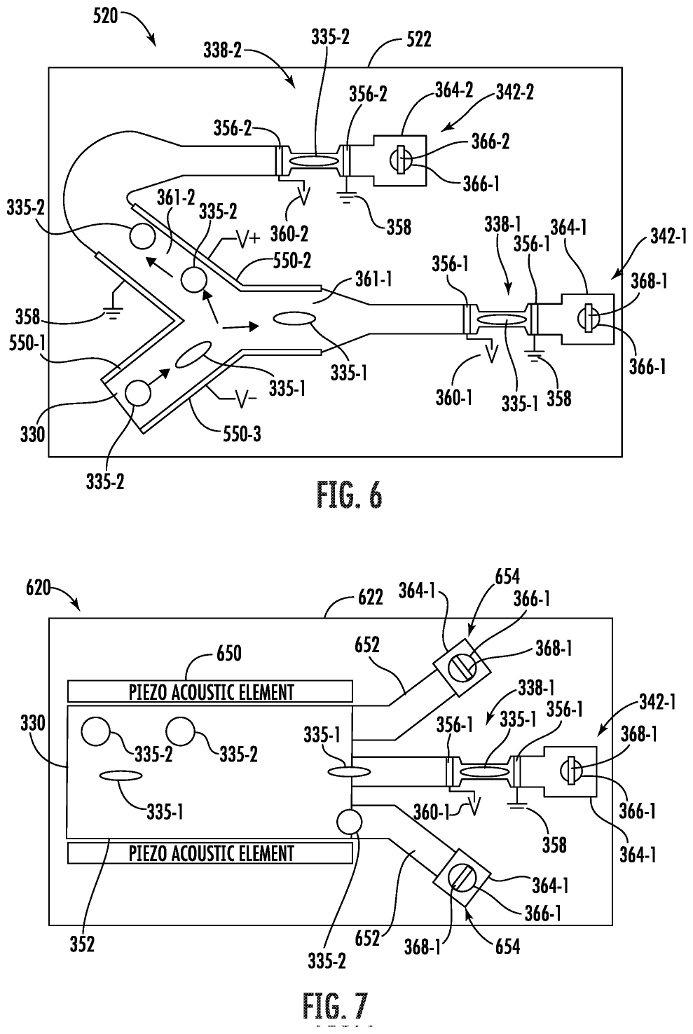

FIG. 6 schematically illustrates portions of an example cell transfection apparatus 520. FIG. 6 illustrates an example of how a microfluidic chip may carry out the sorting of cells using dial electrophoretic cell sorting prior to transfection and dispensing by the same microfluidic chip. Cell transfection apparatus 420 is similar to cell transfection apparatus 320 described above except that cell transfection apparatus 420 comprises a microfluidic chip 522 comprising cell sorter 534 in place of cell sorter 334. Those remaining components of cell transfection apparatus 520 which correspond to components of cell transfection apparatus 320 are numbered similarly.

Cell sorter 534 comprises three electrodes forming a separation or sorting region. Electrode 550-1 comprises an electrode connected to a ground 358. Electrode 550-2 comprises an electrode connected to a positive voltage source. Electrode 550-3 comprise an electrode connected to a negative voltage source. Electrodes 550-3 and 550-2 cooperate to form a first electric field across fluid passage 361-1 which leads to transfection region 338-1. Electrodes 550-1 and 550-2 cooperate to form a second electric field across fluid passage 361-2 which leads to transfection region 338-2. The different electric fields in passages 361-1 and 361-2 exert different dielectrophoretic forces upon the different cells 335-1 and 335-2 such that cells 335-1 are drawn and directed along passage 361-1 while cells 335-2 are drawn and directed along passage 361-2. In one implementation, cells 335-1 may comprise naïve T cells which are being separated are sorted from active T cells 335-2, the naïve T cells being targeted for transfection in region 338-1. In one example of such an implementation, the electric field applied across passages 361-1 and 361-2 is in the range of 10V/mm to 200V/mm. In one example, the fields is 80V/mm. In other implementations, the fields may differ and may have other values depending on the flow rate and the separator geometry. When apparatus 520 is used for sorting other types of cells, other electrical fields may be applied across or within passages 361.

As discussed above, once the cells have been transfected, the cells are dispensed by their respective fluid ejector 342-1 and 342-2. The dispensing of fluid creates a negative fluid pressure so as to draw cells towards fluid ejectors. In some implementations, inertial pumps may be provided on microfluidic chip 522 to assist in the movement of cells 335-1 and 335-2 out of the sorting region and through the transfection region 338 towards the fluid ejectors 342.

FIG. 7 is a schematic diagram illustrating portions of an example cell transfection apparatus 620. FIG. 7 illustrates an example of how a microfluidic chip may carry out the sorting of cells using acoustics prior to transfection and dispensing by the same microfluidic chip. Cell transfection apparatus 620 is similar to cell transfection apparatus 320 described above except that cell transfection apparatus 620 comprises a microfluidic chip 622 comprising cell sorter 534 in place of cell sorter 334. Those remaining components of cell transfection apparatus 520 which correspond to components of cell transfection apparatus 320 are numbered similarly.

Cell sorter 534 comprises an acoustic cell sorter that separates targeted cells from non-targeted cells or other particles using acoustics. Cell sorter 534 comprises piezo acoustic element 650, discharge passages 652 and fluid ejectors 654. Piezo acoustic element 650 surrounds channel or passage 352 and applies a standing surface acoustic wave to passage 352. Cells in the continuous laminar flow through passage 352 may be separated based upon their volume, density and compressibility. Different acoustic forces result in different displacements of cells, repositioning larger cells closer to the center of passage 352 and smaller cells farther from the center.

In the example illustrated, the cells being targeted for transfection, cells 335-1, are larger relative to the non-target cells 335-2. For example, in implementations where cell transfection apparatus 620 is to be used for CAR T therapy, naïve T cells may be different in size than active T cells. Given this relationship between cells 335-1 and 335-2, transfection region 338-1 and fluid ejector 342-1 are generally centered in alignment with the center of passage 352 so as to receive the target cell 335-1 flowing along the center of passage 352. Discharge passages 652 and fluid ejectors 654 are located along the outer periphery of passage 352 to receive the non-target cells 335-2. Fluid ejectors 654 are each similar to fluid ejector 342-1 in that each of fluid ejectors 654 comprises an ejection chamber 364-1, an ejection orifice 366-1 and a fluid actuator 368-1, each of which has been described above. In some implementations, fluid ejectors 654 may be omitted, where reservoirs for collecting and containing the non-target cell 335-2 are provided on microfluidic chip 622.

As should be appreciated, in implementations where the cells being targeted for transfection are smaller than the non-targeted cells, transfection region 33-1 and its associated fluid ejector 342-1 may be located along the outer internal periphery of passage 352 to receive the smaller target cells, whereas discharge passage 652 and its associated fluid ejector 654 may be generally centered in alignment with the center of passage 352 to receive the non-target cells. In some implementations, piezo acoustic element 650 may continuously surround passage 352. In other implementations, piezo acoustic element 650 may intermittently extend about passage 352.

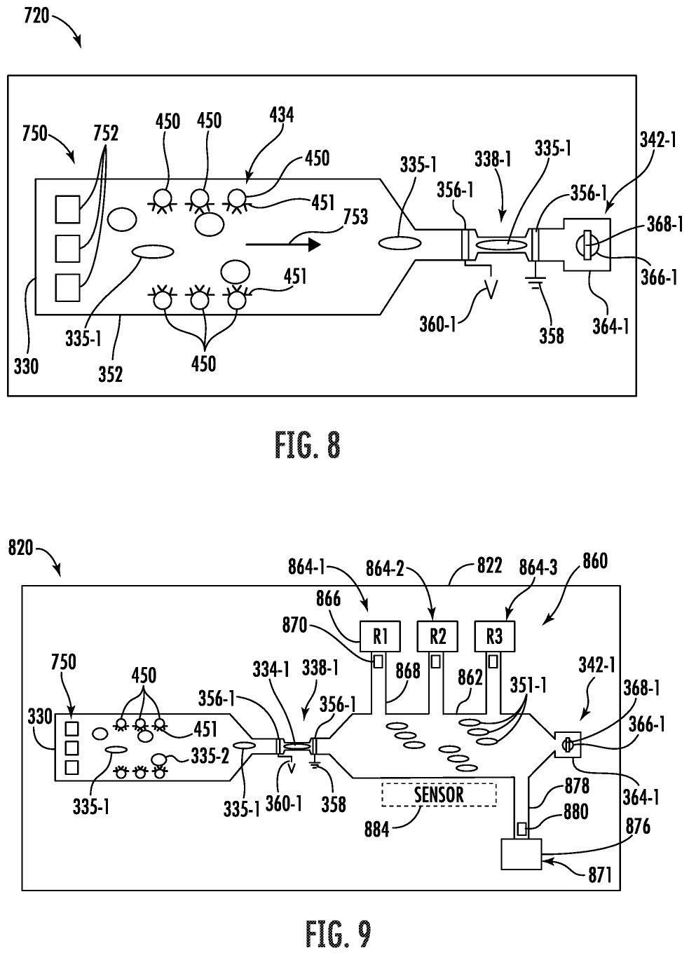

FIG. 8 is a schematic diagram illustrating portions of an example cell transfection apparatus 720. FIG. 8 illustrates an example of how a microfluidic chip may additionally include inertial pumps to move target cells from input port 330, across transfection region 338-1 to fluid ejector 342-1. Cell transfection apparatus 720 is similar to cell transfection apparatus 420 described above except that cell transfection apparatus 620 comprises a microfluidic chip 722 additionally comprising inertial pump 750. Those remaining components of cell transfection apparatus 720 which correspond to components of cell transfection apparatus 420 are numbered similarly.

Inertial pump 750 is located within passage 352 and sizes to move cells along passage 352 towards transfection region 338-1. In the example illustrated in which non-target cells are sorted from cells targeted for transfection using affinity (such as anti-bodies 451 on pillars 450), inertial pump 750 is sized and located such that the displacement forces created by inertial pump 750 are insufficient to detach or separate non-targeted cells from pillars 450. In the example illustrated, inertial pump 750 comprises fluid actuators 752 supported by microfluidic chip 722. In some implementations, fluid actuators 752 comprise thermoresistive elements, such as thermal resistive electrodes, formed along the interior surface of passage 352 so as to move fluid in the direction indicated by arrow 753.

Inertial pump 750 may likewise be used in any of the above described cell transfection apparatus 20, 320, 420, 520 and 620. For example, inertial pump 750 may be located between input port 330 and the particular cell sorter of such apparatus. In some implementations, inertial pump 750 may be located between the cell sorter and the transfection region to pull or draw cells across the cell sorter. In some implementations, the inertial pump 750 may be located so as to also pump or drive cells that have been sorted through and across the transfection region.

FIG. 9 is a schematic diagram illustrating portions of an example cell transfection apparatus 820. FIG. 9 illustrates an example of how cell propagation may be additionally provided on the same microfluidic chip that carries out cell sorting and cell transfection. Cell transfection apparatus 820 is similar to cell transfection apparatus 720 described above except that cell transfection apparatus 820 comprises a microfluidic chip 822 additionally comprising cell propagation region 860. The remaining components of cell transfection apparatus 820 which correspond to components of cell transfection apparatus 720 are numbered similarly.

Cell propagation region 860 is sandwiched between cell transfection region 338-1 and fluid ejector 342-1 on microfluidic chip 822. Cell propagation region 860 comprises cell propagation chamber 862, reagent supplies 864-1, 864-2 and 864-3 (collectively referred to as supplies 864), and lift off reagent supply 871. Cell propagation chamber 862 receives biological cells 351-1 that have been transfected by transfection region 338-1 and contains such cells during their propagation, multiplication or growth. Cell propagation chamber 862 may contain surfaces to which the propagating cells may adhere. In some implementations, cell propagation chamber 862 may comprise small openings connecting the interior of chamber 862 to atmosphere or to gas sources to assist with gas exchange.

Reagent supplies 864 supply reagents such as cell growth media, to chamber 862 to enhance or facilitate the propagation of the transfected cells 351-1 within chamber 862. Each of reagent supplies 864 comprises a reagent source 866 connected to chamber 862 by reagent supply passage 868 that contains an inertial pump 870 to controllably move fluid containing the particular reagent from the reagent source 866 to chamber 862. Reagent source 866 may comprise a reservoir containing a reagent or may comprise a port for connection to an external reagent source. Examples of reagents include, but are not limited to, hi-glucose Dulbecco's Modified Eagle's Medium (DMEM), Roswell Park Memorial Institute (RPMI) culture medium, fetal bovine serum (FBS), non-essential amino acids, RPMI, trace amounts beta-mercaptoethanol, and assorted antibiotics including penicillin and streptomycin.

Lift off supply 871 supplies a lift off reagent to the interior of chamber 862 to assist in the lift off or detachment of the propagated cells 351-1 from the propagation surfaces within chamber 862. Lift off supply 871 comprises lift off reagent source 876 connected to chamber 862 by lift off supply passage 878 which contains an inertial pump 880 to controllably move fluid containing the lift off reagent from the lift off reagent source 876 to chamber 862. Lift off reagent source 876 may comprise a reservoir containing a lift off reagent or may comprise a port for connection to an external lift off reagent source.

As shown by broken lines, in some implementations, microfluidic chip 822 may additionally support a sensor 884. Sensor 884 extends along chamber 862 to sense the interior of chamber 862, facilitating a determination of a state of cell propagation within chamber 862. In some implementations, sensor 884 may comprise an illumination source and a sensor array, such as a liquid crystal display (LCD) sensor array to capture images of the propagating cells 351-1 within chamber 862. In some implementations, sensor 84 may comprise a spectrometer to capture spectrographic information regarding the state of the propagation within chamber 862. In yet other implementations, sensor 84 may be omitted.

Each of the transfection apparatus shown in FIGS. 4-9 may additionally comprise controller 260 (shown in FIG. 3). Controller 260 may be supported by the microfluidic chip that carries out cell sorting, and transfection or may be separate or remote from the microfluidic chip, wherein controller 260 communicates with components of the microfluidic chip in a wired or wireless fashion. Controller 260 may output control signals controlling the fluid ejector(s), the inertial pumps and the supply of power to the electrodes carrying out transfection. Controller 260 may receive signals from sensor 884 or other sensitive determine a state of propagation, wherein controller 260 may control the fluid ejector's, the inertial pumps in the supply of power to the electrodes carry out transfection additionally based upon information obtained from sensor 884. Controller 260 may facilitate an automated process for sorting, transfected (potentially propagating) and dispensing biological cells to a target site, such as individual wells of a well plate. Each of such cell transfection apparatus described in FIGS. 4-9 may be utilized as part of cell propagation system 200 (shown and described with respect to FIG. 3), in place of cell transfection apparatus 20. Each of such cell transfection apparatus 320, 420, 520, 620, 720 and 820 may be contained within an incubator 290, wherein transfected cells are ejected into multi-well plate 250 which is moved by stage 252. In some implementations, the wells deposited within the multi-well plate 250 may be once again imaged by imager 280 and or further propagated in the multi-well plate 250 through the use of media exchange system 254.

Although the present disclosure has been described with reference to example implementations, workers skilled in the art will recognize that changes may be made in form and detail without departing from the disclosure. For example, although different example implementations may have been described as including features providing various benefits, it is contemplated that the described features may be interchanged with one another or alternatively be combined with one another in the described example implementations or in other alternative implementations. Because the technology of the present disclosure is relatively complex, not all changes in the technology are foreseeable. The present disclosure described with reference to the example implementations and set forth in the following claims is manifestly intended to be as broad as possible. For example, unless specifically otherwise noted, the claims reciting a single particular element also encompass a plurality of such particular elements. The terms “first”, “second”, “third” and so on in the claims merely distinguish different elements and, unless otherwise stated, are not to be specifically associated with a particular order or particular numbering of elements in the disclosure.

What is claimed is:

1. A cell transfection apparatus comprising:
a microfluidic chip comprising:
a fluid input port;
a cell sorter to sort target cells from non-target cells in fluid received through the fluid input port;
a cell transfection region comprising an electroporation region to receive the target cells sorted from the non-target cells; and
a fluid ejector to dispense a transfected target cell received from the cell transfection region.

2. The cell transfection apparatus of claim 1, wherein the fluid ejector comprises a fluid actuator selected from a group of fluid actuators consisting of a thermoresistive fluid actuator and a piezo-membrane based fluid actuator.

3. The cell transfection apparatus of claim 1, wherein the microfluidic chip further comprises an inertial pump to move target cells through the cell transfection region.

4. The cell transfection apparatus of claim 1, wherein the microfluidic chip further comprises a cell propagation chamber between the electroporation region and the fluid ejector.

5. The cell transfection apparatus of claim 4, wherein the microfluidic chip further comprises:
a reagent supply passage for connection to a reagent source;
a first inertial pump within the reagent supply passage for pumping cell growth media from the reagent source through the reagent supply passage to the cell propagation chamber; and
a lift off supply passage for connection to a lift off reagent source; and
a second inertial pump within the lift off supply passage for pumping a lift off reagent to the cell propagation chamber.

6. The cell transfection apparatus of claim 1, wherein the microfluidic chip further comprises:
a second cell transfection region comprising a second single cell electroporation region to receive second target cells, different than the target cells, from the cell sorter; and
a second fluid ejector to dispense a transfected second target cell received from the second cell transfection region.

7. The cell transfection apparatus of claim 1, wherein the cell sorter is to sort the target cells from the non-target cells based upon size-elasticity.

8. The cell transfection apparatus of claim 1, wherein the cell sorter is to sort the target cells from the non-target cells using dielectrophoretics.

9. The cell transfection apparatus of claim 1, wherein the cell sorter is affinity based so as to sort the target cells from the non-target cells using negative enrichment of the non-target cells.

10. The cell transfection apparatus of claim 1, wherein the cell sorter comprises an acoustic cell sorter.

11. The cell transfection apparatus of claim 1 further comprising:
a multi-well plate to receive the transfected target cell from the fluid ejector;
a liquid handler to supply a cell growth media to the multi-well plate; and
an incubator containing the microfluidic chip, the multi-well plate and the liquid handler while maintaining temperature and carbon dioxide concentration levels within the incubator.

12. The cell transfection apparatus of claim 11 further comprising an imager to image cells within the multi-well plate.

13. A cell transfection method comprising:
depositing a solution into a port of a microfluidic chip;
sorting a target cell from a non-target cell of the solution with a cell sorter on the microfluidic chip;
moving the target cell through a single cell electroporation region of the microfluidic chip;
transfecting the electroporated target cell on the chip; and
dispensing the transfected target cell from the chip.

14. The cell transfection method of claim 13 further comprising dispensing fluid from the chip to pull the target cell through the single cell electroporation region of the chip.

15. A cell propagation system comprising:
a multi-well plate;
a microfluidic chip comprising:
a fluid input port to receive a fluid containing target cells;
a cell transfection region comprising a single cell electroporation region; and
a fluid ejector to eject droplets of fluid to pull the fluid containing the target cells through the single cell electroporation region;
a liquid handler to exchange media within wells of the multi-well plate;
a stage to position the multi-well plate relative to the fluid ejector and the liquid handler; and
a controller to output control signals controlling the stage, the fluid ejector, and the liquid handler.
